(19) United States
(12) Patent Application Publication
(10) Pub. No.: US 2017/0304579 A1
(43) Pub. Date: Oct. 26, 2017

(54) RADIANT BARRIER FOR HEATED AIR CIRCUITS

(71) Applicant: CareFusion 207, Inc., San Diego, CA (US)

(72) Inventor: Steve LABOLLITA, Rancho Cucamonga, CA (US)

(21) Appl. No.: 15/643,380

(22) Filed: Jul. 6, 2017

Related U.S. Application Data
(62) Division of application No. 12/975,187, filed on Dec. 21, 2010, now abandoned.

(51) Int. Cl. A61M 16/08; A61M 16/10

(52) U.S. Cl. CPC A61M 16/0875; A61M 16/1075; A61M 16/1095; Y10T 29/49826; A61M 2205/0238; A61M 2205/3633

(57) ABSTRACT

A heated breathing circuit with radiant barrier is provided. The breathing circuit includes an airflow conduit configured to receive gas at an input end and configured to deliver the gas to a patient at an output end, a heating element disposed inside the airflow conduit configured to heat the gas inside the airflow conduit between the input end and the output end and a heat shield disposed between the heating element and an outside surface of the airflow conduit such that the heat shield prevents heat energy loss from within the airflow conduit.

### FIELD OF THE INVENTION

The present technology relates generally to the respiratory field. More particularly, the present technology relates to heated breathing circuits.

### BACKGROUND

In general, a breathing circuit is an assembly of components which connects a patient's airway to a machine creating an artificial atmosphere, from and into which the patient breaths. For example, the machine may be a ventilator and the components may be a series of tubes. When the ventilator pushes air through a tube to a patient, the air is sometimes humidified. A heating wire positioned within the tube produces heat that maintains temperature inside the tube to prevent condensation of the humidified air within the tube. Improved breathing circuit heating is desired.

### DESCRIPTION OF EMBODIMENTS

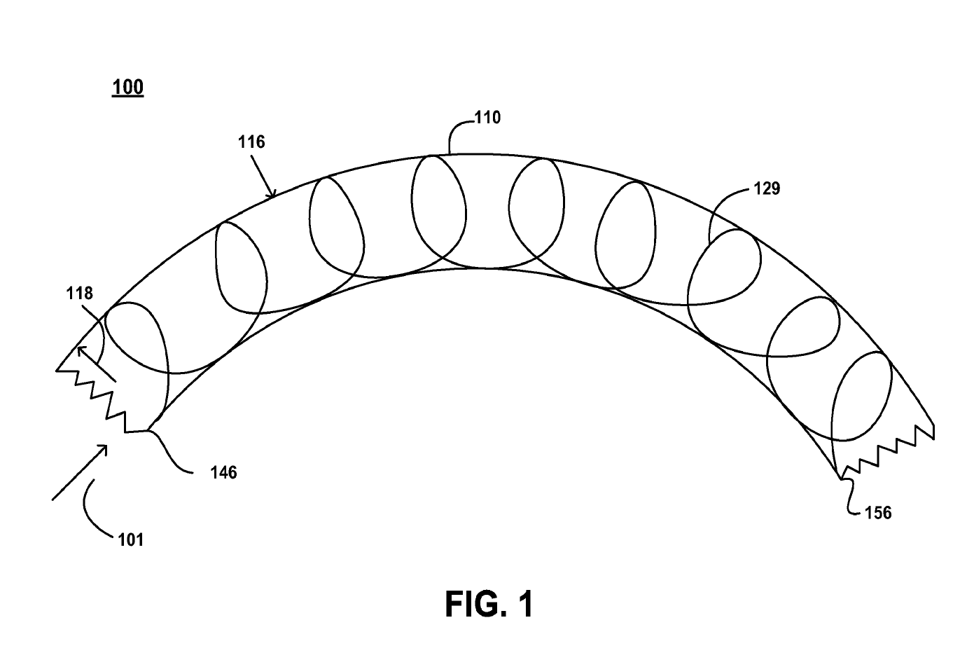

FIG. 1 shows a portion of a breathing circuit in accordance with embodiments of the present invention.

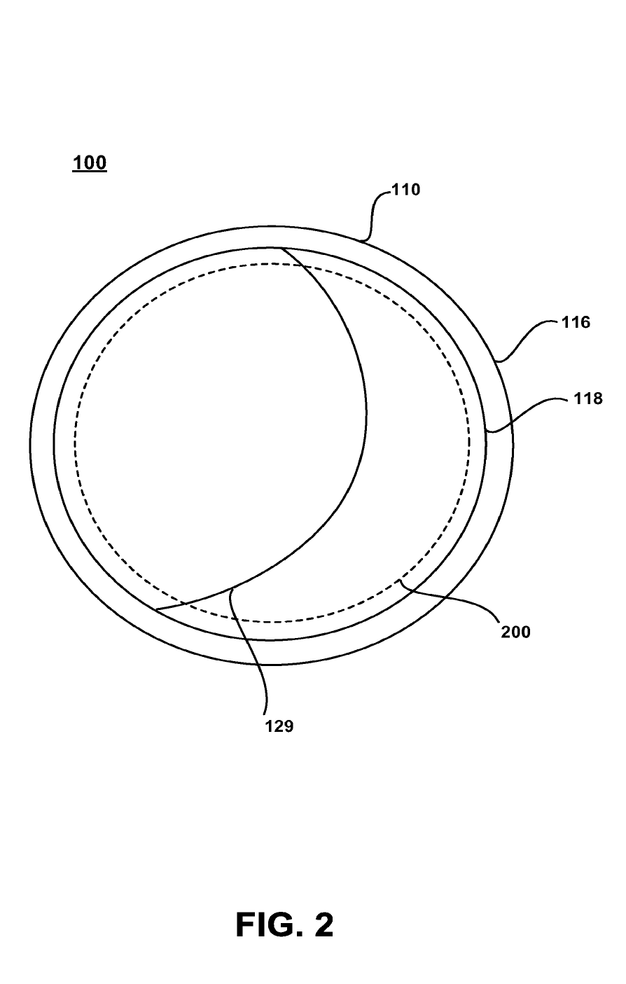

FIG. 2 shows a cross section view of an exemplary breathing circuit including a radiant barrier disposed on an interior surface of the airflow conduit in accordance with embodiments of the present invention.

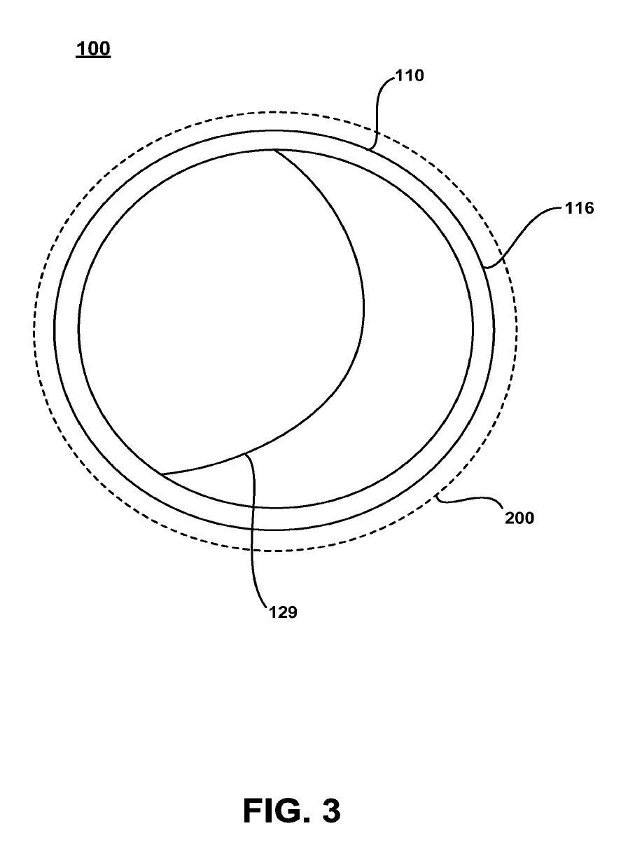

FIG. 3 shows a cross section view of an exemplary breathing circuit including a radiant barrier disposed on an exterior surface of the airflow conduit in accordance with embodiments of the present invention.

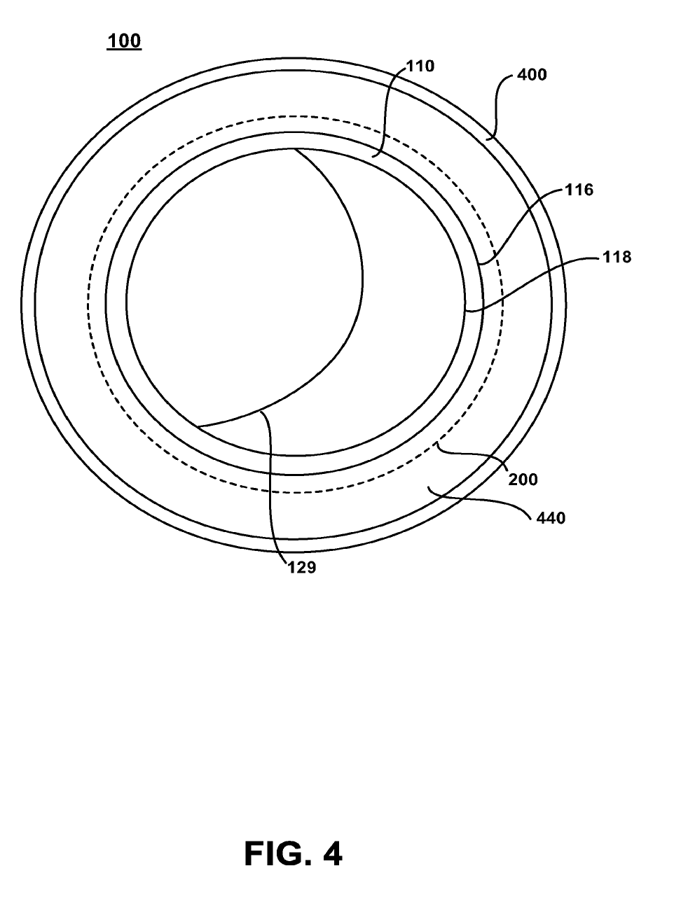

FIG. 4 is a cross section view of an exemplary breathing circuit with an outer insulative conduit and radiant barrier in accordance with embodiments of the present invention.

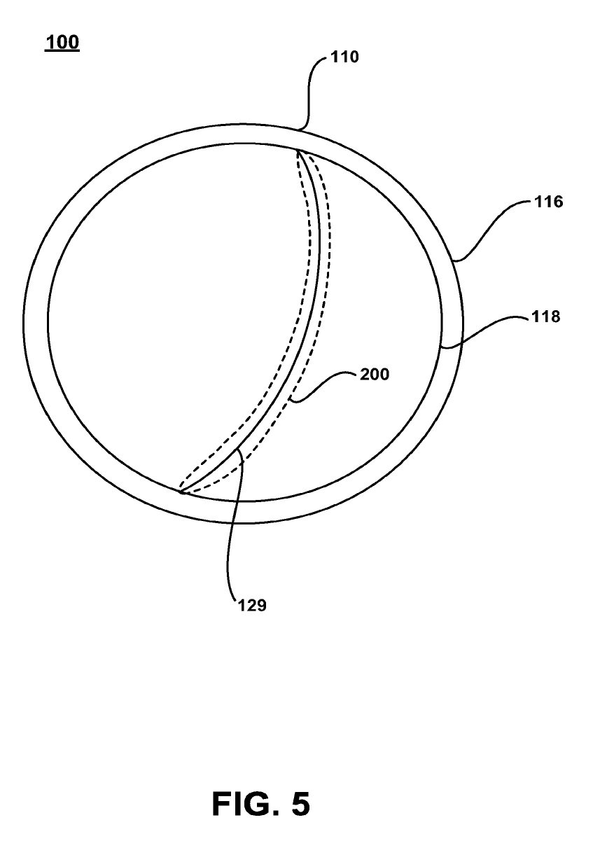

FIG. 5 shows a cross section view of breathing circuit with a radiant barrier on the heating element in accordance with embodiments of the present invention.

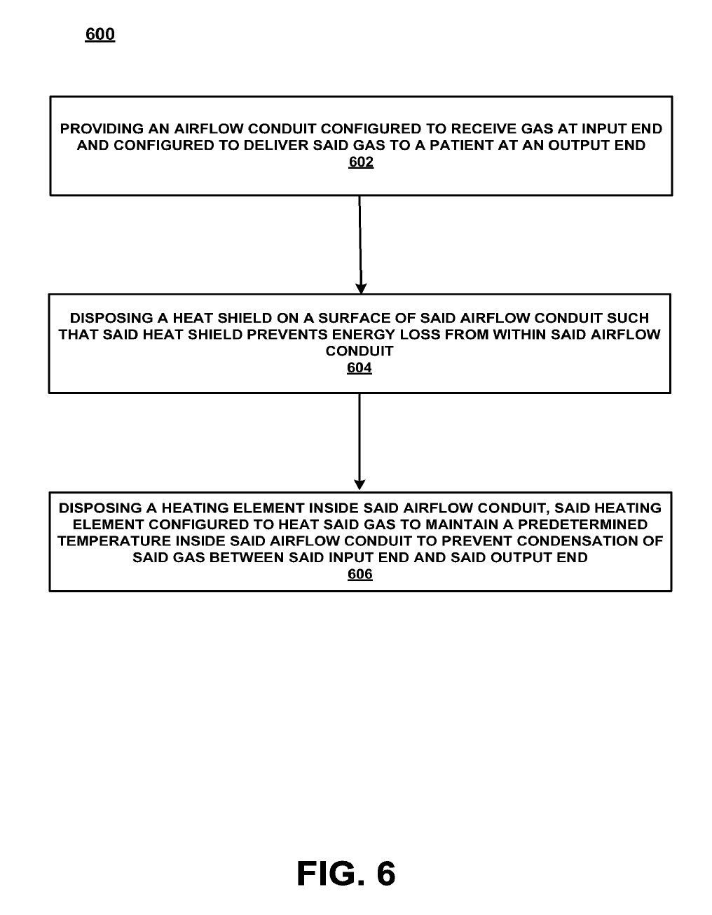

FIG. 6 is a flow diagram of an exemplary method for forming a breathing circuit with a radiant barrier in accordance with embodiments of the present invention.

The drawings referred to in this description should not be understood as being drawn to scale unless specifically noted.

### DESCRIPTION OF EMBODIMENTS

The discussion will begin with an overview of the general use of breathing circuits and the limitations associated therewith. The discussion will then focus on embodiments of the present technology that provide a radiant shield for a heated portion of a breathing circuit.

Breathing circuits are utilized to deliver such medical support as air and anesthetics from a machine that creates an artificial environment to a patient via tubes. Breathing circuits are used in surgical procedures. For example, in a most general case, breathing circuits comprise an inspiratory limb running from a ventilator to a patient and an expiratory limb running from the patient back to the ventilator.

The ventilator pushes air through the inspiratory limb to reach the patient. The patient inhales this pushed air and exhales air into the expiratory limb. For purposes of the present invention, any portion of the breathing circuit could be considered a patient circuit or conduit. It is appreciated that the present invention is well suited to be used in any portion of the patient circuit or any other airflow conduit.

If the air is cold when the patient inhales it, the patient's body works hard to try to warm up the air for ease of breathing. Humidity can also be added to the circuit, because when someone is intubated for ventilation, their upper airways are bypassed. In normal breathing, the upper airways heat and humidify inspired air. Because of the intubation (bypassing upper airways), there is a humidity deficit which creates serious physiological problems if not addressed (e.g., through use of a humidified circuit, or heat and moisture exchanger). When air is humidified, the temperature in the tube must be kept above the dew point to prevent condensation within the tube. Thus, breathing circuits can be designed with heating wires positioned within the interior of at least the inspiratory limb, or patient circuit.

If a heating wire is positioned within the airflow conduit such that the heating wire stretches the full length of the inspiratory limb, then all of the air moving through the inspiratory limb becomes heated. Thus, the air arriving from the inspiratory limb into the patient's airway is also well heated.

The heating wire is an infrared emitter and converts some of the electrical energy to thermal energy through electrical resistance. Water vapor is considered a very good absorber of infrared. Although the conduit of the patient circuit is a thermal insulator, plastics are good absorbers and emitters of infrared. Therefore, the tubing is competing with the water vapor for heat emitted by the wire. Furthermore, the breathing circuit conduit is thin walled and therefore, some heat will be conducted through the wall and emitted (by infrared) to the surrounding environment.

Embodiments of the present invention provide a heated patient circuit with a radiant barrier to trap radiant energy within the patient circuit to improve patient circuit conditions.

FIG. 1 shows a portion of a breathing circuit 100. Breathing circuit 100 is formed from airflow conduit 110 and directs supply gas 101 from an input end 146 to an output end 156 in accordance with embodiments of the present invention. The output end 156 can be coupled with a patient to deliver gas supply 101 to the patient's respiratory system. The input end can be coupled with a gas supply (not shown) that provides gas 101. In one embodiment, gas 101 may be humidified prior to entering the breathing circuit 100 at input end 146.

In one embodiment, the breathing circuit 100 includes a heating wire 129 that is configured to provide heat energy to the gas supply 101. In some cases, gas supply 101 is humidified with water vapor. To prevent condensation of the air supply between the input end 146 and the output end 156, heat is provided by the heating wire 129 to maintain a temperature above the dew point of the air supply 101 which prevents condensation from forming inside the air supply conduit 110.

Although the heating wire is shown as a coil of wire located along the inner cavity of the conduit 110, it is appreciated that any number of heating wire routing options are well suited to be used in accordance with embodiments of the present invention. For example, more than one wire could be used.

Although the surfaces of the airflow conduit are shown as smooth surfaces, it is appreciated that the conduit may not be smooth and may for example, be corrugated to improve flexibility and to prevent line kinking. The radiant barrier of the present invention is well suited to be used with such applications.

Embodiments of the present invention provide a radiant barrier to prevent radiant energy from passing from inside the airflow conduit to the outside environment. The radiant barrier is not shown in FIG. 1 as multiple configurations can be implemented in accordance with the present invention. One or more examples are described below. It is appreciated that any number of configurations of radiant barriers and airway conduit can be used. In one embodiment, a low emissivity material is pre-compounded into the breathing conduit material.

In one embodiment, the radiant barrier is disposed on the interior surface 118 of the airflow conduit 110 to trap the radiant energy within the airflow conduit 110. Although embodiments of the present invention are described in the context of blocking radiant energy, specifically in the infrared range, it is appreciated that embodiments of the present invention could be used to block other heat energy transfer, such as conduction or convective and could be used to block other radiant energy outside of the infrared range.

In one embodiment, the airflow conduit of the present invention includes an outer insulating layer, such as an outer conduit that houses the patient circuit 100. The inner surface of the airflow conduit 110 would include a radiant barrier. It is appreciated that the radiant barrier could be any heat reflective material suitable to be disposed either inside or outside the airflow conduit 110.

For example, the radiant barrier could include metal foil, a metal oxide film or coating, a coated polymer film, a ceramic oxide coating or any other low emissivity material. The radiant barrier of the present invention can be a stand-alone (removable) element of the breathing circuit 100 that can be retrofitted to existing circuits, or can be a coating applied to the circuit itself. The configuration of the radiant barrier can be customized as to minimize any conductive heat loss through the radiant barrier.

FIG. 2 shows a cross section view of en exemplary breathing circuit 100 including a radiant barrier 200 disposed on an interior surface 118 of the airflow conduit 110 in accordance with embodiments of the present invention. In this embodiment, the radiant energy radiated from heating element 129 is blocked by the radiant barrier 200 to prevent the radiation from escaping the airflow conduit 110. In this embodiment, the trapped radiant energy provides heat energy to the gas (not shown) that is being delivered to the patient. The heat energy prevents condensation of the supply gas on the interior surface 118 of the airflow conduit 110. The heat energy also maintains a predetermined temperature of the supply gas to the patient.

The radiant barrier 200 of FIG. 2 may be disposed on the inner surface 118 in any number of ways. For example, the radiant barrier 200 can be formed as a separate removable inner sleeve that is positioned within the airflow conduit 110 prior to the heating element being positioned within the airflow conduit. In another example, the radiant barrier 200 is disposed permanently on the inner surface as a coating or film.

FIG. 3 shows a cross section view of en exemplary breathing circuit 100 including a radiant barrier 200 disposed on an exterior surface 116 of the airflow conduit 110 in accordance with embodiments of the present invention. In this embodiment, the radiant energy radiated from heating element 129 is blocked by the radiant barrier 200 to prevent the radiation from escaping the airflow conduit 110. In this embodiment, the trapped radiant energy provides heat energy to the gas (not shown) that is being delivered to the patient. The heat energy prevents condensation of the supply gas on the interior surface 118 of the airflow conduit 110. The heat energy also maintains a predetermined temperature of the supply gas to the patient. In this embodiment, the radiant barrier 200 may be the outside surface 116 of airflow circuit 110.

The radiant barrier 200 of FIG. 3 may be disposed on the outer surface 116 in any number of ways. For example, the radiant barrier 200 can be formed as a separate removable outer sleeve that is positioned outside the airflow conduit 110. In another example, the radiant barrier 200 is disposed permanently on the outer surface as a coating or film.

FIG. 4 is a cross section view of breathing circuit 100 with an outer insulative conduit 400 in accordance with embodiments of the present invention. In one embodiment of the invention, the airflow conduit 110 is housed within an outer conduit 400.

An air gap 440 provides an insulation layer that further blocks heat energy transfer from the airflow conduit 110. FIG. 4 shows the radiant barrier 200 on an outer surface 116 of the airflow conduit 110, however, it is appreciated that the radiant barrier 200 could also be disposed on the inner surface 118 of the airflow conduit 110. In one embodiment, the air gap 440 is evacuated to further reduce convection heat transfer.

The radiant barrier 200, the air gap 440 and the outer conduit 440 provide insulation for the heat energy generated by the heating element 129 that is housed inside the airflow conduit 110. The improved insulation of heat of the present invention reduces the amount of heat energy that is transferred from inside the airflow conduit 110 to the outside environment which enables improved patient circuit heating. In this embodiment, the infrared shield is disposed between the heating element 129 and the outside surface of the airflow conduit such that said heat shield prevents energy loss from within said airflow conduit.

FIG. 5 shows a cross section view of breathing circuit 100 with a radiant barrier on the heating element 129 in accordance with embodiments of the present invention. In this embodiment, the radiant heat energy is shielded at the heating element 129. In one embodiment, heating wire is coated with the radiant barrier 200. In another embodiment, the heating wire is made from a low emissivity material and does not radiate infrared energy from the heating element. In this embodiment, the heating wire is a poor emitter of infrared radiation and would minimize radiation losses.

FIG. 6 is a flow diagram of an exemplary method 600 for forming a breathing circuit with a radiant barrier in accordance with embodiments of the present invention.

At 602, method 600 includes providing an airflow conduit configured to receive gas at an input end (146 of FIG. 1) and configured to deliver the gas to a patient at an output end (156 of FIG. 1). In one embodiment, the input gas is humidified and comprises water vapor in accordance with embodiments of the present invention.

At 604, method 600 includes disposing a heat shield on a surface of the airflow conduit such that said heat shield prevents heat energy loss from within the airflow conduit. In one embodiment the heat shield is disposed on an interior surface of the airflow conduit. In another embodiment, the heat shield is disposed on an exterior surface of the airflow conduit. In another embodiment, the heat shield is disposed between an interior surface of the airflow conduit and an exterior surface of the airflow conduit, for example, within the airflow conduit material.

At 606, method 600 includes disposing a heating element inside the airflow conduit, the heating element configured to heat the gas inside the airflow conduit to maintain a predetermined temperature of the gas and to prevent condensation of the gas inside the airflow conduit between the input end and the output end.

All statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present technology is embodied by the appended claims.

1-31. (canceled)

32. A breathing circuit, comprising:
an airflow conduit configured to receive gas at an input end and configured to deliver the gas to a patient at an output end; and
an infrared emitting heating element disposed in the airflow conduit and having a radiant barrier formed on the infrared emitting heating element,
wherein said radiant barrier is configured to shield radiant heat energy from the infrared emitting heating element at the infrared emitting heating element such that the radiant barrier prevents absorption of infrared radiation from the heating element by the airflow conduit.

33. The breathing circuit of claim 32, wherein said heating element is configured to heat the gas to maintain a predetermined temperature inside the airflow conduit to prevent condensation of the gas between the input end and the output end.

34. The breathing circuit of claim 32, wherein the radiant barrier comprises a coating on the infrared emitting heating element.

35. The breathing circuit of claim 34, wherein the coating comprises a metal oxide material.

36. The breathing circuit of claim 34, wherein the coating comprises a polyester film.

37. The breathing circuit of claim 34, further comprising an outer conduit that houses the airflow conduit.

38. The breathing circuit of claim 37, further comprising an evacuated air gap between the outer conduit and the airflow conduit.

39. The breathing circuit of claim 32, wherein the airflow conduit is formed from a thermally-insulating plastic.

40. A breathing circuit, comprising:
an airflow conduit configured to receive gas at an input end and configured to deliver the gas to a patient at an output end; and
a heating wire disposed in the airflow conduit and configured to heat the gas to maintain a predetermined temperature inside the airflow conduit to prevent condensation of the gas between the input end and the output end,
wherein the heating wire is made from a low emissivity material and does not radiate infrared energy from the heating wire.

41. The breathing circuit of claim 40, further comprising an outer conduit that houses the airflow conduit.

42. The breathing circuit of claim 41, further comprising an evacuated air gap between the outer conduit and the airflow conduit.

43. The breathing circuit of claim 40, wherein the airflow conduit is formed from a thermally-insulating plastic.

44. The breathing circuit of claim 40, wherein the airflow conduit comprises an inspiratory limb, and wherein the heating wire stretches a full length of the inspiratory limb.

45. A method for forming a breathing circuit, the method comprising:
providing an airflow conduit configured to receive gas at an input end and configured to deliver the gas to a patient at an output end; and
providing, in said airflow conduit, an infrared emitting heating element having a radiant barrier formed on the infrared emitting heating element,
wherein said radiant barrier is configured to shield radiant heat energy from the infrared emitting heating element at the infrared emitting heating element such that the radiant barrier prevents absorption of infrared radiation from the heating element by the airflow conduit.

46. The method of claim 45, further comprising providing an outer conduit for housing the airflow conduit.

47. The method of claim 46, further comprising forming an evacuated air gap between the outer conduit and the airflow conduit.

48. The method of claim 45, wherein the heating element is configured to heat the gas to maintain a predetermined temperature inside the airflow conduit to prevent condensation of the gas between the input end and the output end.

49. The method of claim 45, wherein providing the infrared emitting heating element comprises forming a coating on the infrared emitting heating element.

50. The method of claim 49, wherein the coating comprises a metal oxide material.

51. The method of claim 49, wherein the coating comprises a polyester film.
